(19) United States
(12) Patent Application Publication
(10) Pub. No.: US 2026/0253505 A1
(43) Pub. Date: Aug. 27, 2026

(54) SYSTEMS AND METHODS FOR PROFILING COGNITIVE ABILITIES

(71) Applicant: Yiannis Laouris, Washington DC, DC (US)

(72) Inventor: Yiannis Laouris, Washington DC, DC (US)

(21) Appl. No.: 19/063,370

(22) Filed: Feb. 26, 2025

(51) Int. Cl. G09B 5/02; G09B 5/04

(52) U.S. Cl. CPC G09B 5/02; A61B 5/4088; G09B 5/04; G16H 50/20

(57) ABSTRACT

A system and method for measuring and assessing cognitive abilities of a user are disclosed. The method includes generating an individualized assessment protocol comprising a plurality of tasks each configured to evaluate cognitive mental specification. The method also includes the user selecting at least one task tasks using a semi-random process. The method further includes displaying a task-specific interface to present the selected task to user. The method includes receiving user responses to the task along with associated parameters. The method includes transmitting an individualized performance score table to a server. The method also includes processing the individualized performance scores using a population database, a neural network classifier, and regression models, which adjust their parameters based on the received scores. The method further includes computing individualized assessment scores to generate a profile of cognitive mental abilities. The method also includes transmitting profile of cognitive mental abilities to user device.

### FIELD OF THE INVENTION

The present invention relates to cognitive psychology and technology-based assessments of cognitive disorders, including dyslexia, learning disabilities, and comorbidities, leveraging digital platforms and algorithmic task generation for individualized cognitive profiling.

### BACKGROUND OF THE INVENTION

Existing methods of scientifically exploring and describing the brain include sciences such as neuroanatomy, neurophysiology and neuropharmacology. When it comes to studying brain function in particular, the classic and older approaches used by trained neurologists, comprised mainly of studying the responses of sensory and/or motor organs connected to the brain to specific stimuli (e.g., elicited or voluntary movements, reflexes, sensations, etc.). These methods usually involve a specialist and they require so much time that renders it impossible to apply to a large number of individuals.

More recently, new approaches to studying the brain emerged, such as those neuroimaging techniques that build up a picture of the brain based on the differential absorption of X-rays (e.g., Computed tomography (CT) scanning), or those that record magnetic resonance imaging (MRI), or measure brain activity by detecting changes in blood oxygenation and flow that occur in response to neural activity (Functional magnetic resonance imaging; fMRI), etc. Aforesaid methods are highly specialized, extremely expensive and typically reserved for patients at high risk and/or those who already have a diagnosis.

Several tests to evaluate visual-or auditory memory, discrimination and processing, sequencing, categorization, lateralization, and other mental abilities and correlated those measurements to other measurements that have been traditionally used to evaluate cognitive performance including, but not exclusively, IQ tests, reading test, Raven's Progressive Matrices, WISC tests, etc. Several research indicate that drill and practice or other methods and tools that aim to improve such cognitive skills may also improve performance in real-world abilities including those that relate to practical skills such as driving, sports, or carrying out other activities of daily living, etc. Other studies have shown that improving underlying cognitive skills may improve critical thinking, performing well in school, exams, and at work. Even though it is evident that having an exact profile of such cognitive skills is a necessary requirement, a suitable and accessible method to acquire such a profile is not available. Such a profile may also enhance the capabilities of educators to adapt their teaching to the individual abilities, learning styles and needs of each child, as the current educational trend demands.

Besides tests that evaluate intelligence and speed of processing, certain combinations thereof as well as few specifically developed with the aim to diagnose dyslexia and/or other learning conditions and/or difficulties, there are virtually no automated, computer-based, comprehensive tests that can produce a precise profile of cognitive mental abilities, within a short time and efficiently.

The United States Publication No. U.S. Pat. No. 6,164,975A discloses an interactive instructional system that adapts teaching strategies based on a learner's cognitive profile but relies on predefined instructional content. It lacks dynamic, real-time task generation and AI-driven profiling for comprehensive cognitive assessment.

The United States Publication No. US20160364377A1 discloses a system that integrates data from multiple sources to facilitate precision medicine, focusing on compiling and cross-comparing medical data stored in separate silos to improve data retrieval and analysis. This system primarily addresses the challenges of data integration and computational efficiency in medical research.

The United States Publication No. U.S. Pat. No. 3,095,653A discloses a method of testing learning by allowing instructors to input correct answers into a system, which then transmits this information for assessment purposes. It lacks algorithmic task generation and AI-driven profiling to dynamically assess cognitive abilities.

The United States Publication No. US20100035225A1 discloses an adaptive spaced teaching method that enhances long-term retention by delivering test items at intervals, customizing future delivery based on learner interactions. This approach focuses on improving recall through spaced repetition without employing real-time task generation or AI techniques.

The United States Publication No. US20120208169A1 discloses a system and method for assessing cognitive abilities using computer-based tasks, focusing on evaluating specific cognitive functions through standardized tests. This approach primarily utilizes static tasks without real-time adaptation or personalized profiling.

The United States Publication No. US20120322043A1 discloses an adaptively spaced repetition learning system that presents material at intervals to facilitate learning over time, adjusting future presentations based on user performance. This method focuses on enhancing retention through spaced repetition without employing real-time task generation or AI techniques.

The United States Publication No. US20150228197A1 discloses a system and method for adaptive learning that personalizes educational content delivery based on a learner's performance and engagement metrics. This approach focuses on tailoring instructional materials to individual learning styles and progress but does not dynamically generate cognitive assessment tasks or adjust task difficulty in real-time based on user responses. Additionally, it lacks AI-driven profiling mechanisms, such as neural networks and regression models, for comprehensive evaluation across multiple cognitive domains, and does not ensure culture-independent assessments.

The United States Publication No. US20160092788A1 discloses a system and method for adaptive learning that personalizes educational content delivery based on a learner's performance and engagement metrics but lacks real-time cognitive assessment through algorithmic task generation. The system primarily adapts instructional content rather than evaluating specific cognitive functions such as auditory memory, sequencing, visual memory, and lateralization ability.

The United States Publication No. US20160155345A1 discloses a cloud-based adaptive learning platform (ALP) designed to support educational mobile or web applications. This platform focuses on delivering personalized educational content by adapting to a user's learning pace and style.

The United States Publication No. US20160225274A1 discloses a system and method for providing personalized educational content based on a user's performance and engagement metrics. This approach focuses on adapting instructional materials to individual learning styles and progress.

The United States Publication No. US20130018960A1 discloses an online system that enables group interaction around shared content, facilitating collaborative engagement among users. This system focuses on providing a platform for multiple users to interact with the same content simultaneously.

The United States Publication No. US20130204873A1 discloses a system and method for adaptive learning that personalizes educational content delivery based on a learner's performance and engagement metrics. This approach focuses on providing instructional materials to individual learning styles and progress.

The United States Publication No. US20160219006A1 discloses a method for replacing typed emoticons with user photos in electronic communications, enhancing personalization by substituting textual emoticons with corresponding images from the user's photo library. This approach focuses on enriching user interaction by incorporating personalized visual elements.

The United States Publication No. U.S. Pat. No. 9,116,509B2 discloses rhythm-based brain fitness processes and systems designed to enhance cognitive functions through rhythm cognitive training games. These games are intended to be simple to learn and interact with, aiming to lower the barrier to entry for adults.

The United States Publication No. US20130323704A1 discloses systems and methods for assessing fluid intelligence through matrix reasoning assessments (MRAs). These MRAs present users with matrices containing patterns and missing elements, requiring them to identify the correct design to complete the pattern.

The prior arts cited above lack algorithmic task generation and AI-driven profiling to dynamically assess cognitive abilities in real-time, instead focusing on predefined instructional adaptation, spaced repetition, or static assessments. They do not utilize neural networks and regression models to refine individualized cognitive profiles, nor do they offer semi-random task selection, adaptive difficulty adjustments, or real-time performance scoring. Additionally, they fail to provide personalized, scalable, and culture-independent evaluations across multiple cognitive domains, restricting their applicability to structured learning rather than comprehensive cognitive assessment. Furthermore, despite a plethora of available paper tests, there are very few computerized tools for diagnosing dyslexia and other learning difficulties while simultaneously assessing various components of cognitive performance, which could reveal underlying weaknesses in reading, test performance, school, or work. There are also no tools available to differentiate primitive cognitive abilities and correlate them with real-world performance, creating a major challenge for educators, healthcare providers, and parents.

Therefore, there is a need for a computerized system that can not only diagnose dyslexia and other learning difficulties but also assess various components of cognitive performance in real time. Such a system should incorporate algorithmic task generation, AI-driven profiling, and adaptive difficulty adjustments to provide a personalized and scalable cognitive evaluation. Most importantly, there is a need for a solution that can differentiate primitive cognitive abilities and correlate them with real-world performance, addressing the challenges faced by educators, healthcare providers, and parents in understanding and improving cognitive skills.

### SUMMARY OF THE INVENTION

It is an objective of the present invention to provide a system and method for remotely measuring, profiling, and assessing cognitive mental abilities using algorithmic task generation and computing technologies.

It is another objective of the present invention to enable precise and repeatable cognitive assessments through dynamically generated task sequences tailored to individual user responses.

It is yet another objective of the present invention to develop a technology-driven approach for evaluating cognitive mental abilities independent of language and cultural factors.

It is further objective of the present invention to generate a comprehensive cognitive profile by analyzing response accuracy, completion times, and inter-response decision patterns.

It is another objective of the present invention to facilitate large-scale, cost-effective cognitive evaluations using web-based, mobile, and standalone computing platforms.

It is yet another objective of the present invention to provide real-time adaptability in cognitive testing by dynamically adjusting task difficulty based on user performance.

It is further objective of the present invention to enable demographic-based comparative cognitive profiling across age, gender, family, educational background, and other relevant attributes.

It is another objective of the present invention to capture and process data related to auditory memory, auditory and visual discrimination, sequencing ability, visual memory, categorization ability, and lateralization ability.

It is yet another objective of the present invention to provide a standardized yet customizable platform for use by individuals, educators, psychologists, and medical professionals.

The disclosed invention addresses numerous challenges outlined in the background section. In an exemplary embodiment, the present invention provides a platform that collects previously unavailable datasets in both nature and volume. The system captures user responses, decision-making patterns, selection trends, and movement patterns, and integrating these data with demographic attributes. The platform subsequently analyzes the collected data to generate a cognitive profile for each user, which may be evaluated individually or comparatively against other users within defined groups, including but not limited to, individuals of the same age, gender, family, school, or other relevant demographic segments. This results in a comprehensive cognitive mental ability profiling system.

According to an embodiment of the present invention, a method for measuring and assessing cognitive abilities of a user is disclosed. The method includes generating, by a computing system, an individualized assessment protocol comprising a plurality of tasks, wherein each task configured to evaluate at least one cognitive mental specification. The method also includes transmitting individualized assessment protocol to a user device. The method further includes the user selecting at least one task from the plurality of tasks using a semi-random process. The method includes displaying a task-specific interface to present the selected task to the user. The method also includes receiving user responses to the task along with associated parameters, including timestamps. The method further includes formatting the response data into an individualized performance score table suitable for transmission. The method includes transmitting the individualized performance score table to a server. The method also includes processing, by the server, the individualized performance scores using a population database, a neural network classifier, and regression models, which adjust their parameters based on the received scores. The method further includes computing, by the neural network classifier and regression models, individualized assessment scores to generate a profile of cognitive mental abilities. The method also includes transmitting the profile of cognitive mental abilities to the user device.

According to an embodiment of the present invention, a system comprising a processor and memory, wherein the processor and memory in combination are operable to implement a method for measuring and assessing cognitive abilities of a user is disclosed. The method includes generating, by a computing device, an individualized assessment protocol comprising a plurality of tasks, wherein each task configured to evaluate at least one cognitive mental specification. The method also includes transmitting individualized assessment protocol to a user device. The method further includes the user selecting at least one task from the plurality of tasks using a semi-random process. The method includes displaying a task-specific interface to present the selected task to the user. The method also includes receiving user responses to the task along with associated parameters, including timestamps. The method further includes formatting the response data into an individualized performance score table suitable for transmission. The method includes transmitting the individualized performance score table to a server. The method also includes processing, by the server, the individualized performance scores using a population database, a neural network classifier, and regression models, which adjust their parameters based on the received scores. The method further includes computing, by the neural network classifier and regression models, individualized assessment scores to generate a profile of cognitive mental abilities. The method also includes transmitting the profile of cognitive mental abilities to the user device.

### BRIEF DESCRIPTION OF THE DRAWINGS

The novel features of the disclosure are described with particularity in the claims, which were presented above. A better understanding of the features can be obtained by reference to the following more technical description of illustrative embodiments of how the principles of the disclosure are utilized, and the accompanying drawings of which:

FIG. 1 is a schematic view of a representative example of the logic device through which assessment of a profile of cognitive mental abilities can be achieved.

FIG. 2 shows a schematic view that exemplifies the communication environment.

FIG. 3A depicts an example of a visual interface comprising of a simple numeric keypad presented to the user during an auditory memory task.

FIG. 3B depicts further examples of numeric keypads inspired from telephone or other numeric data entry devices on which the user is expected to enter the numbers imposed by the task at hand.

FIG. 4A depicts an example with two cartoon type humans which “speak” a word, or play the sound of a letter or word.

FIG. 4B depicts further examples of three visual representations of the sound playing device in which the word played, is symbolized by text-like characters which are non-existent in any language known to the user.

FIG. 4C depicts further examples of visual representations of played sounds which might be based on existing objects that are known to be associated with certain sounds, such as the sound made by someone eating, or someone canoeing in the sea, or an alarm ringing or any other physical object that makes a characteristic sound, and the user has to associate the sound with the object.

FIG. 4D depicts metaphors of talking character which may be symbolized, as creature 460, and/or a geometric object 470, and/or loudspeakers 480, and/or any abstract object in 2D, black and white or color 490.

FIG. 5A depicts an example of a digital display interface for the realization of a sequencing test.

FIG. 5B depicts a visual representation of a story, with a beginning, snapshots from the evolution of a story in between and an ending, and a user expected to rearrange the representations of the events in the order they have happened, etc.

FIG. 6A depicts an example of a visual interface presented to the user during a visual memory task.

FIG. 6B depicts further examples of optional visual interfaces presented to the user during visual memory tasks, such as the cards may be placed with some overlapping between them, the images behind the cards may be similar and not the same.

FIG. 7A depicts a digital display interface for assessing the categorization ability with three environments and an object which has to be sorted by drugging it to the most relevant one, wherein the example shows a pencil which belongs in an office environment and not in the bathroom or the bedroom.

FIG. 7B depicts further embodiments in which the environments are a sea, a yard and the sky.

FIG. 8A depicts a digital display interface for testing the lateralization ability.

FIG. 8B depicts the situation when the person is facing the user and the objects maybe different.

FIG. 9 is a flow diagram that exemplifies the flow of processes within a chosen tasks targeting the measurement and assessment of a specific cognitive mental ability.

FIG. 10 is a flow diagram that exemplifies the logic and the flow of processes that lead to the calculation of the user's performance, presents the results of the assessment to the user via a visually displayed interface and provides options for mailing and/or printing and/or sharing with family and/or experts.

### DETAILED DESCRIPTION OF THE INVENTION

The present disclosure relates to a system and a method leveraging modern technological solutions to enable widespread accessibility and facilitating the testing and assessment of cognitive mental abilities in a large population at a low cost. The method evaluates brain function by administering a series of cognitive assessments that are independent of language and culture, conceptualizing the brain as a system—akin to a machine or a black box—that exhibits measurable specifications. It is hypothesized that these measurable specifications correspond to fundamental cognitive mental abilities that underlie higher-order functions, including learning, information processing, critical thinking, and the development of strategies for survival and success. By determining these specifications, the method provides data that may assist in diagnosis, inform targeted interventions, and facilitate the design of individualized learning programs to enhance cognitive performance. The profiling of cognitive mental abilities (PCMA) enables an effective evaluation and prediction of cognitive performance in individuals.

The present disclosure further provides systems and methods for measuring, profiling, and assessing cognitive mental abilities. These systems and methods allow users to assess their own cognitive abilities or those of others, including children or patients, through remote computing technologies in a manner that ensures precision and repeatability. In some embodiments, the assessment methodology employs algorithmic techniques to randomize task generation in real time, thereby dynamically presenting the next set of tasks to the user. Each task is categorized according to cognitive domain and subdomain, the difficulty level appropriate for the user, and other relevant task-specific parameters. The approach by the present invention facilitates accurate comparisons across multiple testing sessions, benchmarking user performance against various populations, and dynamically adjusting the difficulty level based on user responses. The computer-based nature of the assessment allows for implementation over the internet and enables access through standard web browsers, smart mobile devices (e.g., iPhone, Android phone), or smart tablets (e.g., iPad, Google Pixel, Microsoft Surface, Samsung Galaxy, Sony Xperia). Any computing system capable of displaying visual content, receiving user input, locally storing data, executing mathematical computations, and transmitting data to a cloud-based system may be utilized for implementing the method.

In one embodiment, the method is implemented within an interactive multimedia environment, such as HTML5, Flash, or similar web-based technologies, for execution within web browsers or smart device interfaces. In another embodiment, the method is implemented in standalone interactive environments, including UNITY, SHOCKWAVE, and other development tools designed for platform-specific execution.

In an exemplary embodiment, the disclosed a system and a method utilizing a plurality of visual display interfaces to measure, profile, and assess various cognitive mental abilities. These interfaces are designed to evaluate a range of cognitive functions, including but not limited to:
a. Auditory Memory test
b. Auditory and Visual Discrimination
c. Sequencing test
d. Visual Memory test
e. Categorization ability test
f. Lateralization ability test

Scientific definitions of the cognitive mental abilities assessed by the disclosed methods are provided herein to facilitate an understanding of the operation of the visual display interfaces.

Auditory Memory: The cognitive ability to receive orally presented information, process it, retain it, and subsequently recall it (Bellis, 2003; Roeser & Downs, 2004; Stredler-Brown & Johnson, 2004).

Auditory Discrimination: A fundamental auditory processing skill that involves the ability to differentiate among phonemes, which are the smallest meaningful units of sound in a language. This ability is vital for language comprehension and speech recognition.

Visual Discrimination: The ability to recognize and differentiate visual details, including but not limited to shapes, forms, colors, and spatial positioning of objects, people, and printed materials. This ability is essential for reading, writing, and object recognition.

Sequencing Ability: The cognitive skill of arranging elements in a logical order, such as reconstructing a segmented object (e.g., head, body, tail) or retelling events in the correct chronological sequence (e.g., beginning, middle, and end of a story). It also encompasses the recognition of ordered processes, such as the sequential stages of an activity (e.g., eating an apple—whole, partially eaten, core remaining).

Visual Memory: The ability to retain and recall the characteristics of an object or visual stimulus. Impairments in this ability may affect reading comprehension and overall information processing, as information must first be stored in short-term memory before transitioning to long-term memory.

Categorization Ability: The cognitive process by which objects, concepts, or information are recognized, classified, and grouped based on shared attributes. Categorization facilitates efficient information retrieval and problem-solving by identifying commonalities among diverse stimuli.

Lateralization Ability: The identification and differentiation of one side of the body from the other, as well as recognizing left or right hand dominance. This ability extends to spatial awareness, including the capacity to recognize and adjust to a body part's rotation by 180°.

The assessment methodology employs algorithmic processing of user responses to tasks presented via dedicated visual display interfaces. The assessment metrics include, but are not limited to, the total number of correct responses, response times for individual tasks, time taken to complete individual subtasks, and total completion time for all subtasks at a given level and/or across multiple levels within a visual display interface corresponding to a specific cognitive mental ability.

The systems and methods described herein rely on a variety of computer systems, networks and/or digital devices for their operation. In order to fully comprehend and appreciate the operation of the system, a basic understanding of suitable computer systems useful. The systems and methods disclosed herein are enabled as a result of application via a suitable computing system.

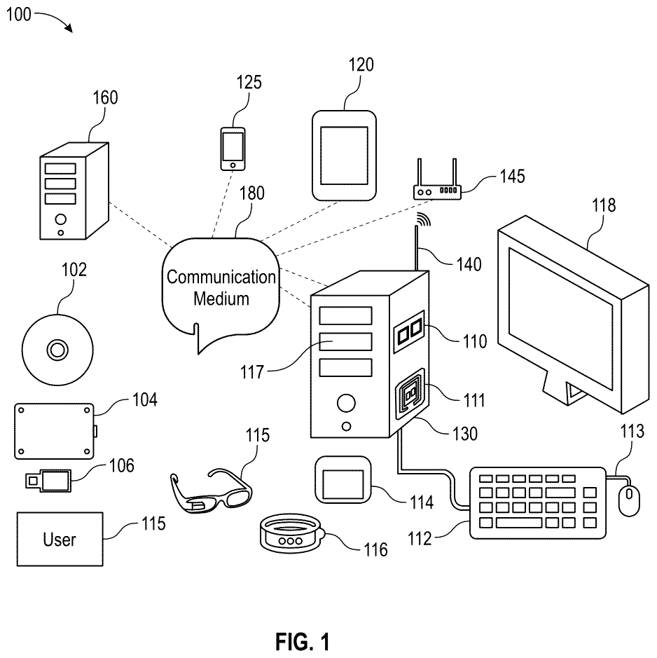

According to an embodiment of the present invention, FIG. 1 shows a representative example of a system through which assessment of the profile of cognitive mental abilities can be achieved when a logic device is used to access a browser through which the present invention is implemented. A computer system (or other equivalent digital device) 100, which may be understood as a logic apparatus capable of reading instructions from digital media such as a CD 102, an external disk drive 104, a USB stick 106 and/or network port 110, is connected to a server 160. The computer system 100 can be connected to the internet and/or an intranet. The system includes a Central Processing unit (CPU) 111, optical CD or other disk drives 117, optional input devices, illustrated as keyboard 112 and/or mouse 113 and/or mouse track 114 and/or Google glasses or equivalent 115, and/or hand mounted posture input devices 116 and a monitor 118.

The communication medium 150 can include any suitable means of transmitting and/or receiving data. For example, the communication medium can be comprised of an ethernet-based network connection 110 and/or a wireless connection 140 to a nearby Wi-Fi apparatus 145 and/or a wireless connection to the internet via a mobile device 125 or other GSM G3/G4/G5 120 or other connection. It is envisioned that data produced through the invention disclosed herein can be transmitted bi-directionally over such networks and/or connections. The computer system 100 can be capable, or at least in some situations communicating with a user device 120, 125 or 130, used by a user. The computer system 100 is also capable of communicating bi-directionally with other computer systems over an intranet and/or the internet, and/or with other computer systems via the server 160.

The internet uses a protocol called TCP/IP (i.e., Transmission Control Protocol/Internet Protocol) to connect computer systems 100 from which some serve as hosts and others as access points. The infrastructure is controlled by Internet Service Providers (ISPs). The Internet Protocol (IP) enables data to be sent from one device (e.g. a computer system, a mobile phone, a smart digital device etc.) to another device on a network. Each device has at least one IP address, which serves as a unique identifier when the device communicates with other devices or hosts. The communication does not need to be synchronous or continuous. When a device sends or receives data or messages, the data or messages are separated into packets, each packet is monitored by the protocol until it reaches its destination, even if it takes a different path. The unique IP and the protocol ensure the fidelity of the transmission.

Contemporary devices can communicate with one another and with computer system networks wirelessly, using diverse protocols and systems. Cellular and wireless telephones use RFC, GSM or equivalent technologies and protocols, home and office devices connect to a home or office based system called access point through a WiFi protocol. In many of the configurations of the innovation disclosed here, the user accesses digital content through a software called the browser, connected to the front end server via a network, which is typically the Internet, but which could also be any type of wired or wireless, private or public network. Large numbers of users can be in communication with the front end at any given time. The user may access the front end through a variety of computing devices 130, 120 or 125, such as personal computers, cellular or other mobile phones, smart digital devices etc.

The browser allows users to access content hosted in web pages on the World Wide Web using any type of relevant applications. Such applications suitable for the purpose of the innovation disclosed herein, include, but are not limited to, Microsoft Internet Explorer, Mozilla Firefox, Apple Safari, Google Chrome, or any other application capable of or adaptable to allowing access to web pages on the World Wide Web. The browser can also include a video player (e.g., Quicktime™ from Apple, Flash™ from Adobe Systems, Inc.), or any other player adapted for the video file formats used in the video hosting website. Alternatively, videos can also be accessed using a standalone program.

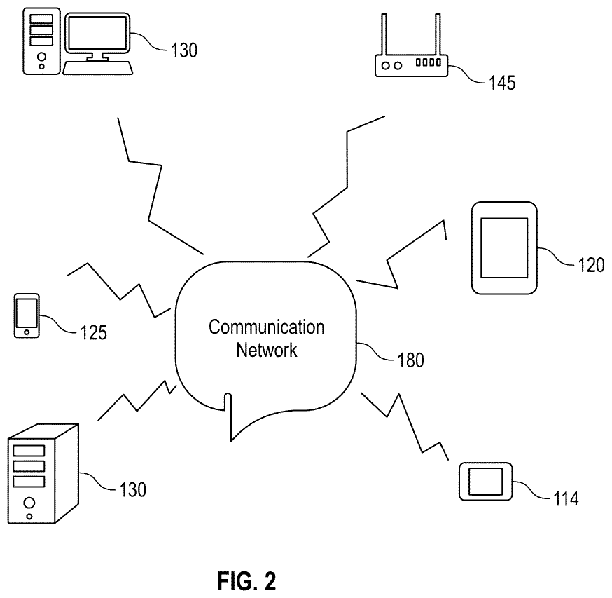

The desired technical effect and assessment of the user's profiling is achieved through the computing system 100, described in FIG. 1, which is part of a communication network 180. As shown in FIG. 1, a server 160 may be interconnected with the computer system 100 via a communications network 180. As shown in FIG. 2, the communication network environment 180 may comprise of, or being a combination of a fixed-wire or wireless intranet, extranet, peer-to-peer network, virtual private network, the Internet, or other communications network technologies and protocols with a number of client computing environments such as a personal computer 130, smart phone 125, and/or other personal digital devices 120/122 using any of a number of known protocols, such as, hypertext transfer protocol (HTTP), file transfer protocol (FTP), or wireless application protocol (WAP). Additionally, the communication network 180 can utilize various data security protocols such as secured socket layer (SSL) or pretty good privacy (PGP).

When the system is operating, a user (via user device 130) may interact with a computing application running on a client computing environment to access and or produce and/or transmit desired data. The computing applications and/or produced data may be stored the server 160 and communicated to users through client computing environments over the communications network 180. The computing applications, described in more detail herein, are used to achieve the desired technical effect and assessment. A user may request access to specific applications and/or data housed in whole or in part on the server 160. These data may be communicated between client computing environment for processing and storage. The server 160 may host computing applications, processes and applets for the generation, authentication, encryption and communication between applications and data and/or may cooperate with other servers (not shown in the schematics), third party service providers (also not shown in the schematics), and storage area networks (SAN) to realize application/data transactions.

Utilizing the foregoing computing environment, the Profiling of Cognitive Mental Abilities (PCMA) is achieved by measuring and evaluating an array of cognitive mental abilities of a plurality of users. The method disclosed uses visual display interfaces to measure, profile and assess respective cognitive mental abilities. The cognitive mental abilities being assessed and profiled include, but not limited to, auditory memory, auditory and visual discrimination, sequencing ability, visual memory, categorization ability and lateralization ability.

In some aspects, the measurement of each cognitive mental ability comprises of presenting the user with subtasks, which are grouped together to form levels, and the system recording the user's responses. The presentation of subtasks could be randomized so as to present a group of subtasks for the purpose of measuring a cognitive mental ability, followed by a presentation of subtasks for the purpose of measuring another cognitive mental ability and so forth. In some aspects, the sequence, number and duration of subtasks may be calculated dynamically and is adapted to the previous responses of the user. The present invention disclosed herein uses algorithmic methods randomizing next-task generation that allow real-time creation of the next tasks to be presented to the user. These items are classified by the domain and subdomain of the cognitive mental type which is targeted, the degree of difficulty with which the user should be confronted to resolve the task and other characteristics relevant to the specific subdomain under consideration. This allows for accurate comparison across retests, comparisons of the user's performance in relation to other populations, as well as for adapting the degree of difficulty of the assessment session to the responses of the user.

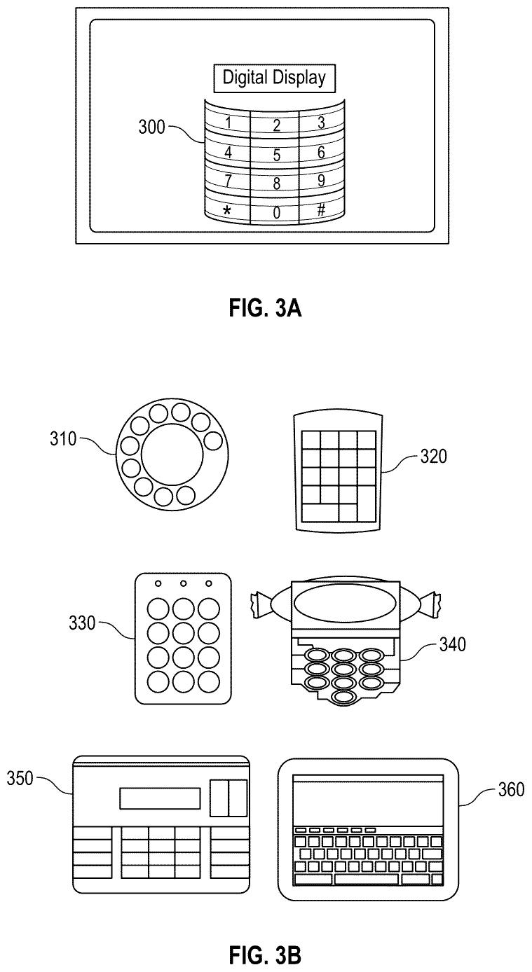

In this aspect, the visual display interfaces used to measure, profile and assess distinct respective cognitive mental ability are described. In an auditory memory test, the system 100 plays, through the speakers of the user device 120, 125, 130 that the user is using to interact with the system 100, a sequence of numbers and subsequently the system 100 requests the user, using auditory or visual methods, to repeat the sequence by pressing the numbers in the visual display in the order played. For example, the task is embedded within metaphors including but not limited to, calling a friend using a telephone, entering data into a security panel to open a door, or the like. The user is asked to repeat sequences with 1, 2, 3, 4, 5, and/or more digits. For each case, the user is presented subtasks with the same number of digits more than a few times, wherein all these tasks grouped together in what is called a “level”. The keys representing numbers are arranged as depicted in FIG. 3A, or FIG. 3B, i.e. simple numeric keypad 300, classic dial pads 310, classic numeric pads as on keyboards 320, small key-size numeric pads 330, space-like embodiments 340, security panels 350, 360 or any other configuration, including digital display embodiments with symbols instead of simple numbers, and/or including a randomized order in their presentation to the user.

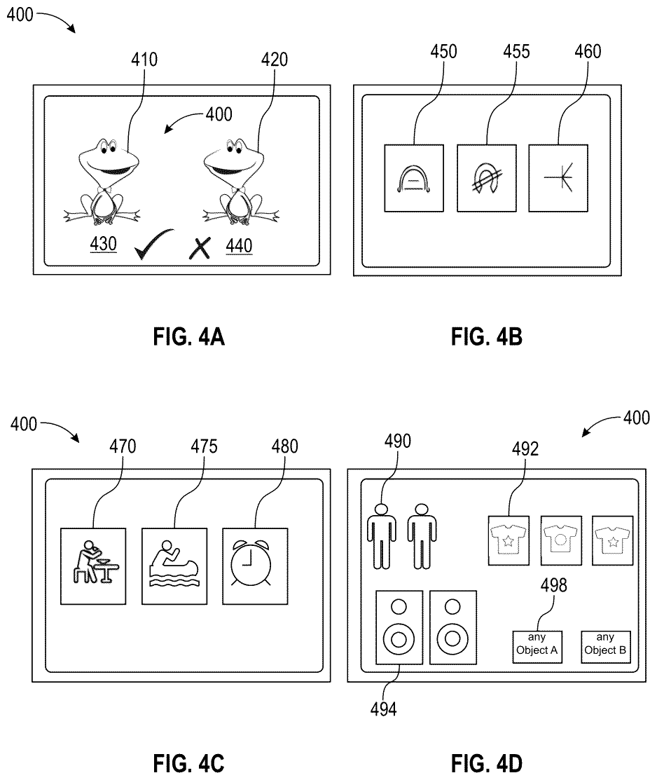

In another aspect, the auditory and visual discrimination test is depicted in FIG. 4A as an example of a digital display interface 400 with two cartoon type humans 410, 420, which speak a word, or play the sound of a letter or word from a given alphabet or language or play the sound of a word non-existent in any language known to the user comprising of one or more syllables. The talking character may or may not animate while the sound is produced. The user has to respond by pressing the yes button 430 if they two characters speak the same sound or the no button 440 if they do not. FIG. 4B depicts further examples of three visual representations of the sound playing device in which the word played 450, 455, 460, is symbolized by text-like characters which are non-existent in any language known to the user. FIG. 4C depicts further examples of visual representations of played sounds which are based on existing objects that are known to be associated with certain sounds, such as the sound made by someone eating 470, or someone canoeing in the sea 475, or an alarm ringing 480 or any other physical object that makes a characteristic sound, and the user has to associate the sound with the object. The talking character may be symbolized, as shown in FIG. 4D by any other representation of a creature 490, and/or a geometric object 492, and/or loudspeakers 494, and/or any abstract object in 2D, black and white or color 498.

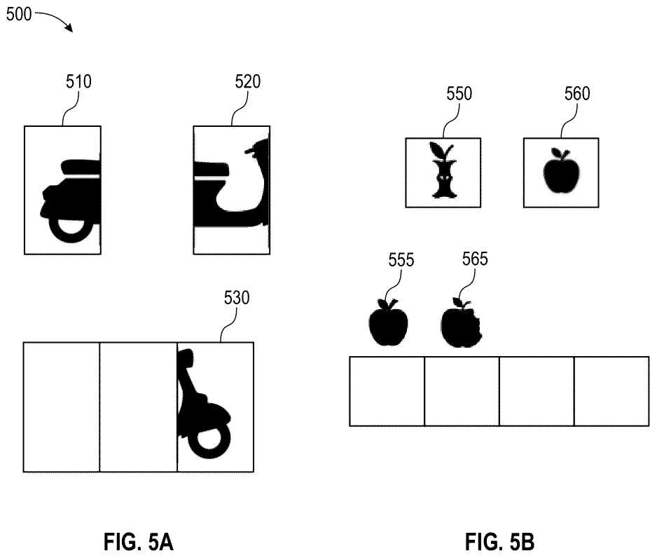

In another embodiment, FIG. 5A depicts an example of a digital display interface 500 for the realization of a sequencing test. In this embodiment, the user is presented with objects which are separated in parts and/or dissected 510, 520, 530 and is requested to put the parts in a logical sequence. Another embodiment, FIG. 5B might consist of a visual representation of a story, with a beginning, snapshots from the evolution of the story in between and an ending, and the user expected to rearrange the representations of the events in the order they have happened, etc. The example depicts eating an apple 550, 555, 560, 565 is shown here only to illustrate a process, but any thinkable process may be used as a metaphor to test a user of her or his ability to arrange the events in a story in the right sequence.

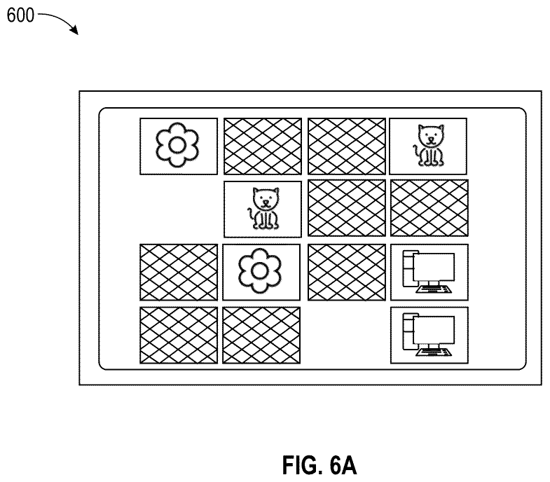

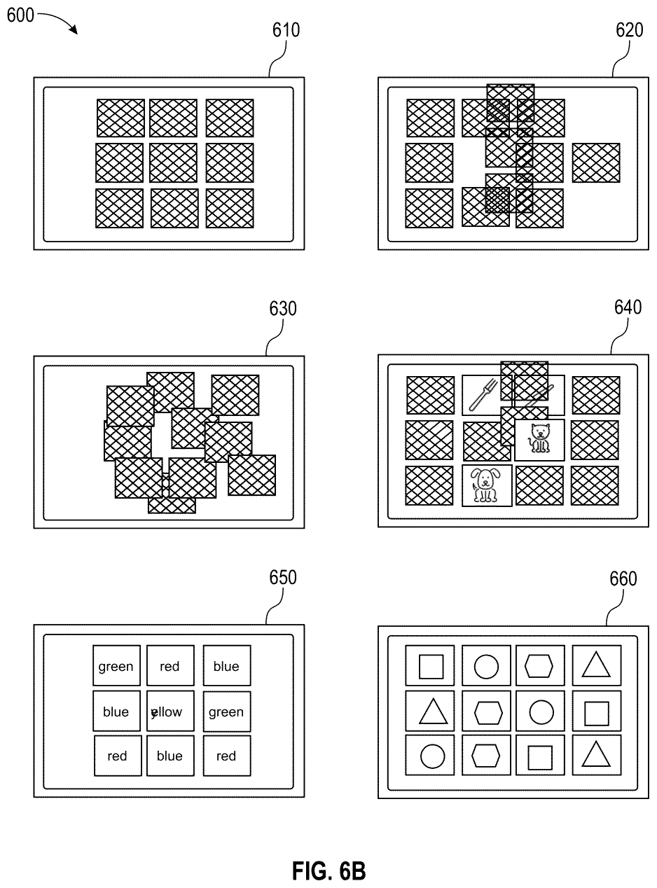

Referring FIGS. 6A and 6B depict a digital display interface of a visual memory test 600 similar to a cards game, and the user is expected to turn two cards and if they have the same image underneath, they either go away 605 or remain turned 608. The embodiment of the game disclosed herein includes tasks for which instructions may be given to the user visually and/or also via recorded voice and the images may be identical (e.g. two identical images of a dog), similar (e.g. images of two different dogs), or related in some property (e.g. images of two animals; images of two flowers, etc). The number of cards placed on the digital display interface 610 may vary and/or some cards 620 may be missing, the cards 630 may be placed with some overlapping between them, the images behind the cards may be similar and not the same, i.e., conceptually related, implying they can be correlated like for example a glass and a plate, or a boy and a girl, a dog and cat like in 640, the cards may not have realistic objects but geometric objects 660 or colors 650 or other combination hereof and/or the backside of the card may also have color, objects or other symbols as in 640, 650 and 660.

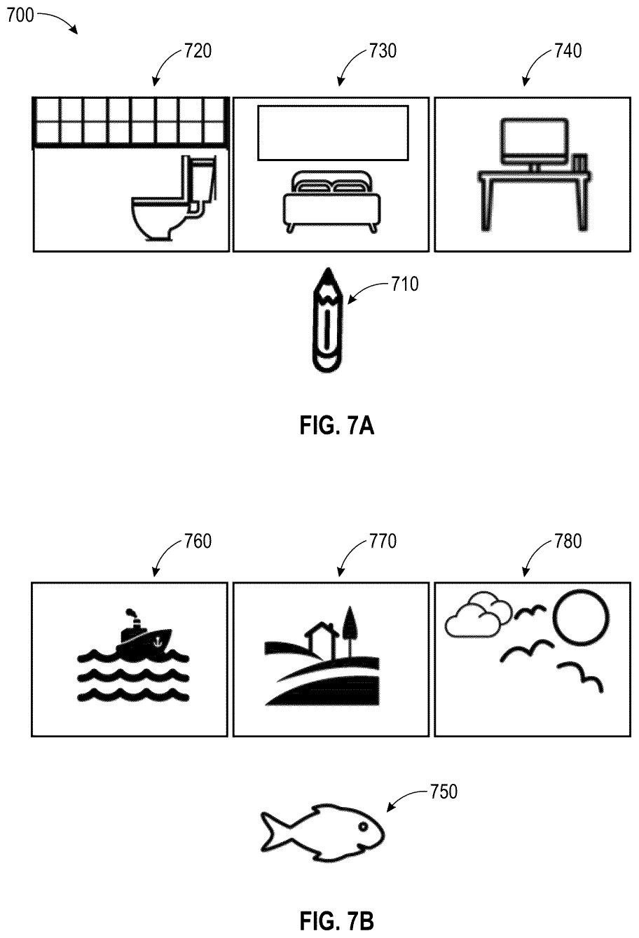

In another embodiment, a digital display interface of the categorization ability test 700 presents the user three environments and an object which has to be sorted by dragging it to the most relevant one. The example in FIG. 7A shows a pencil 710 which belongs in an office environment 740 and not in the bathroom 720 or the bedroom 730. The second embodiment shown in FIG. 7B depicts three different environments, a sea 760, a yard 770 and the sky 780. A fish 750 is expected to be moved into the sea 760.

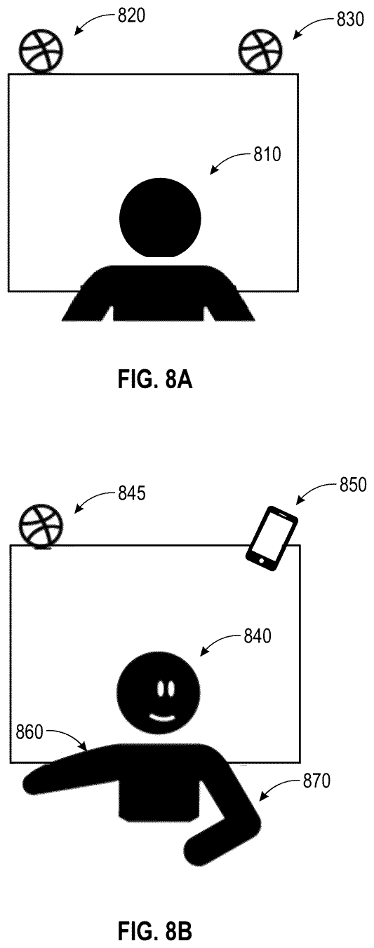

A digital display interface of a lateralization ability test depicted in FIG. 8 presents the user with an abstract person facing a shelf 810 on which two objects appear, one to his left 820 (here as example a ball) and one to his right 830. The user is given instructions to grab either the object on her/his left or right by clicking first the correct object and then the correct arm (860 and 870 in FIG. 8B). The example in FIG. 8A shows the person 810 while in FIG. 8B the person 840 is facing the user. The objects maybe the same as in the case 820 and 830 or different as in 845 and 850.

In an exemplary aspect, the user sends, via their user devices 120, 125, 130, their responses to each task presented along with associated parameters, including timestamps. Further, user's responses to each task are scored based on predefined scoring criteria to generate first individualized performance scores by the computer system 100. In an aspect, the first individualized performance scores are formatted into an individualized performance score table suitable for transmission to the server 160. In some aspects, the server transmits the first individualized performance scores for processing by a population database, a neural network classifier, and regression models which adjust their parameters based on the received scores. Subsequently, the server transmits second individualized performance scores to neural network classifier and regression models to compute revised individualized assessment scores to generate a profile of cognitive mental abilities. The first individualized assessment scores include the latest individualized assessment scores. The second individualized assessment scores include the previous individualized assessment scores. In one aspect, the first and second individualized assessment scores are compared by the neural network classifier and regression models to generate the revised individualized assessment scores, based on which the profile of cognitive mental abilities of the user is generated. For instance, when the second individualized assessment scores are not available, then the first individualized assessment scores is determined to be the revised individualized assessment scores. In some aspects, the neural network classifier and regression models continuously process incoming assessment data, adjusting their parameters based on both individual performance and population-level statistics. Thus, the assessment remains calibrated across different user populations and over time. Upon generating, the profile of cognitive mental abilities is transmitted to the user device.

According to an embodiment of the present invention a method for assessing cognitive abilities through a computerized system is disclosed. The computing system 100 is the system disclosed in aforementioned embodiments. Although the following method presents the preferred embodiments of the invention, it should be understood that modifications and variations are possible without departing from the spirit and scope of the invention.

The method is implemented using the computing system 100 discussed in above embodiments, for protocol generation and management, a user device for task presentation and response collection, and a server equipped with processing capabilities including neural network classifiers and regression models. These components operate within a secure communications environment, utilizing encrypted protocols for both wired and wireless data transmission to ensure the integrity and confidentiality of assessment data.

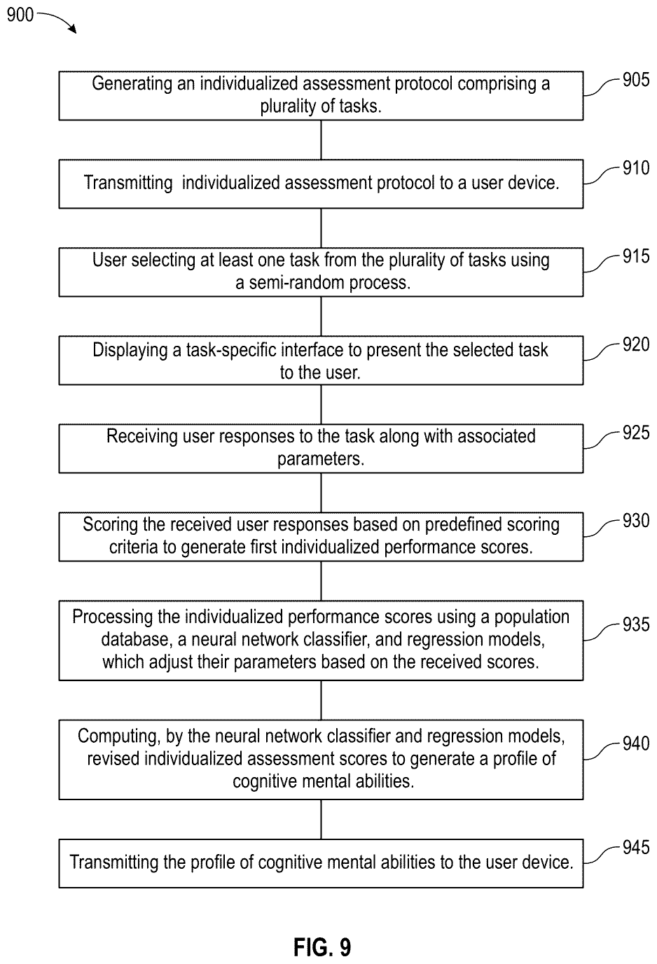

As illustrated in FIG. 9, the method 900 begins with the generation 905 of an individualized protocol by the computing system 100. The protocol consists of multiple cognitive tasks, each designed to evaluate specific mental capabilities. The protocol generation process considers various factors including any previous assessment history, age-appropriate difficulty levels, and the need for task variety to maintain user engagement. The system implements adaptive sequencing based on performance, ensuring that the assessment remains both challenging and informative.

Once generated, the assessment protocol is transmitted 910 to the user device through secure channels. The system employs a semi-random task selection step 915 by the user to maintain unpredictability while ensuring comprehensive coverage of all targeted cognitive abilities, such that it helps prevent learning effects that might skew assessment results while maintaining the validity of the evaluation. In some aspects, the tasks are presented 920 through a task-specific interface for specific cognitive assessments. In an exemplary embodiment, the plurality of tasks includes tasks targeting a different cognitive mental ability. The tasks comprises game-based challenges involving ordered and/or randomized sequences of subtasks assessing cognitive mental abilities including auditory memory, auditory and visual discrimination, sequencing, visual memory, sequencing ability, and lateralization ability.

In one embodiment, for auditory memory evaluation, the interface includes a dynamic numeric keypad with variable arrangements, geometric configurations, and orientation options. These variations prevent pattern memorization while assessing both memory and spatial processing capabilities. The response timestamps and patterns are recorded for detailed analysis.

In some embodiments, in the assessment of auditory and visual discrimination, the interface incorporates engaging elements such as animated talking characters, non-linguistic symbols, and familiar objects associated with specific sounds. These elements are presented through the interface that maintains user engagement while collecting precise response data. Both accuracy and response timing are tracked to build a comprehensive picture of the user's discrimination abilities.

In some embodiments, for sequencing ability assessment, users interact with interfaces displaying dissected objects, story-based image sequences, and multiple environment sorting tasks. These challenges incorporate various elements including color coding, geometric relationships, and orientation-based sorting exercises. The computing system 100 analyzes both the accuracy of sequence reconstruction and the approach taken by the user to understand their sequential processing capabilities.

In some embodiments, for visual memory assessment, interface displays two-dimensional arrays of shapes with varying arrangements, missing elements, and overlapping components. Users engage with abstract design matching exercises and pattern recognition tasks that evaluate both immediate and short-term visual memory capabilities. The computing system 100 tracks not only correct matches but also the patterns of errors to provide insight into visual processing strengths and weaknesses.

In some embodiments, the assessment of lateralization ability employs interfaces with abstract person representations and shelf arrangements requiring left/right discrimination. Users complete object selection and arm movement coordination tasks that evaluate spatial awareness and body schema understanding. In an aspect, both the accuracy and response patterns to assess lateralization development.

At step 925, the user responses associated with the task are received along with associated parameters, including timestamps. Further, the received user responses are scored 930 based on predefined scoring criteria to generate first individualized performance scores. The first individualized performance scores are formatted into an individualized performance score table suitable for transmission. The individualized performance score table is transmitted to the server. The first individualized performance scores are processed 935 using a population database, a neural network classifier, and regression models, which adjust their parameters based on the received scores. Further, revised individualized assessment scores are computed 940, by the neural network classifier and regression models, to generate a profile of cognitive mental abilities. In some aspects, the computing includes transmitting second individualized performance scores to neural network classifier and regression models to compute revised individualized assessment scores. The first individualized assessment scores include the latest individualized assessment scores while second individualized assessment scores include the previous individualized assessment scores. In one aspect, the first and second individualized assessment scores are compared by the neural network classifier and regression models to generate the revised individualized assessment scores, based on which the profile of cognitive mental abilities of the user is generated. For instance, when the second individualized assessment scores are not available, then the first individualized assessment scores is determined to be the revised individualized assessment scores. In some aspects, the neural network classifier and regression models continuously process incoming assessment data, adjusting their parameters based on both individual performance and population-level statistics. Thus, the assessment remains calibrated across different user populations and over time. Upon generating, the profile of cognitive mental abilities is transmitted 945 to the user device.

The profile incorporates both current performance metrics and historical data to provide a comprehensive view of cognitive development and capabilities. The results are presented through the user device in an understandable format that highlights both strengths and areas for potential development. The entire process is monitored and managed through secure communications protocols, maintaining data integrity and user privacy throughout the assessment lifecycle.

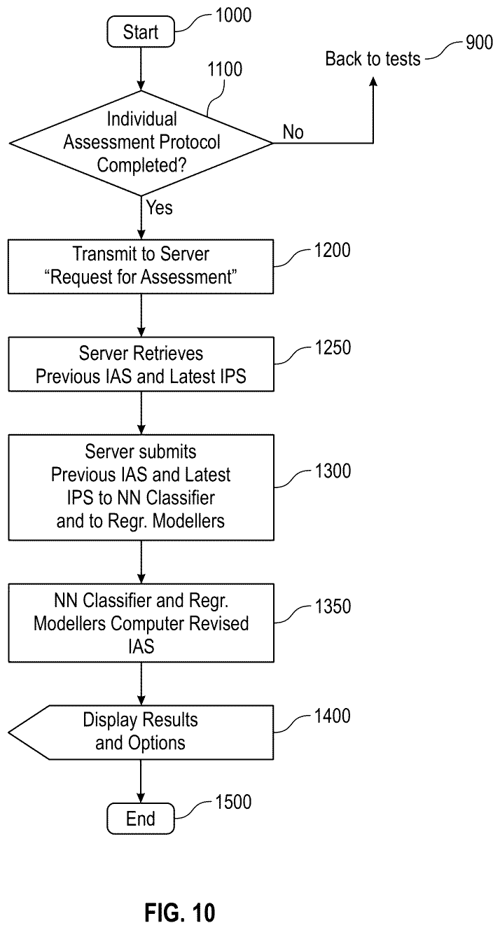

FIG. 10 exemplifies the processes 1000 that lead to the calculation of the user's Individual Assessment Scores (IAS), present the results of the assessment to the user 1400 and provide options for mailing and/or printing and/or sharing with family and/or experts. First, it is checked whether the Individual Assessment Protocol (IAP) has been completed 1100. If not, then the process jumps back to tests 900. Otherwise, the computing system 100 from which the user accesses the system transmits 1200 a “Request for Assessment” to the Server 160. The server retrieves 1250 any previous IAS and the latest IPS, and submits 1300 them to NN Classifier and to Regression Modellers, which in turn compute 1350 revised the revised IAS for the user. Server transmits the revised IAS to users device, which displays and allows the user to mail and/or print and/or sharing the data and terminates 1500.

1. A method for measuring and assessing cognitive abilities of a user, the method comprising:
generating, by a computing system, an individualized assessment protocol comprising a plurality of tasks, wherein each task configured to evaluate at least one cognitive mental specification;
transmitting individualized assessment protocol to a user device;
user selecting at least one task from the plurality of tasks using a semi-random process;
displaying a task-specific interface to present the selected task to the user;
receiving user responses to the task along with associated parameters, including timestamps;
scoring the received user responses based on predefined scoring criteria to generate first individualized performance scores;
processing, by a server, the individualized performance scores using a population database, a neural network classifier, and regression models, which adjust their parameters based on the received scores;
computing, by the neural network classifier and regression models, revised individualized assessment scores to generate a profile of cognitive mental abilities; and
transmitting the profile of cognitive mental abilities to the user device.

2. The method of claim 1, wherein the plurality of tasks includes tasks targeting a different cognitive mental ability, the tasks comprising:
game-based challenges involving ordered and/or randomized sequences of subtasks assessing cognitive mental abilities including auditory memory, auditory and visual discrimination, visual memory, sequencing ability, and lateralization ability.

3. The method of claim 2, wherein the assessment of auditory memory is performed via a numeric keypad interface of the user device, through which the user submits responses to the assessment system for evaluation.

4. The method of claim 2, wherein the assessment of auditory memory includes displaying variant numeric keypads with differing key geometries, positions, and orientations, and receiving user responses for evaluation.

5. The method of claim 2, wherein the assessment of auditory and visual discrimination includes presenting an interface with talking character metaphors, symbolized by non-linguistic characters, existing objects associated with specific sounds, loudspeakers, abstract objects, geometric shapes, or creatures, and receiving user responses for evaluation.

6. The method of claim 2, wherein the assessment of sequencing ability includes presenting an interface displaying dissected objects or images representing a story with a beginning, middle, and end, and receiving user responses for evaluation.

7. The method of claim 2, wherein the assessment of visual memory includes presenting an interface displaying a two-dimensional array of rectangles, squares, cubes, or polygons, which may have varying arrangements, missing elements, overlapping components, abstract designs, colors, or symbols, wherein users match identical, similar, or related images to complete the task.

8. The method of claim 2, wherein the assessment of sequencing ability includes presenting an interface displaying two or more environments and one or more objects, requiring the user to sort objects into the most relevant environment based on color, geometry, orientation, or physical space representation.

9. The method of claim 2, wherein the assessment of lateralization ability includes presenting an interface displaying an abstract person facing a shelf with objects on the left and right, requiring the user to select an object and the corresponding arm to complete the task.

10. The method of claim 1, wherein the computing comprising:
transmitting, by the server, second individualized performance scores to neural network classifier and regression models; and
comparing the first and second individualized assessment scores by the neural network classifier and regression models to generate the revised individualized assessment scores;
wherein when the second individualized assessment scores are not available, then the first individualized assessment scores is determined to be the revised individualized assessment scores.

11. The method of claim 10, wherein the first individualized assessment scores include the latest individualized assessment scores and the second individualized assessment scores include the previous individualized assessment scores.

12. The method of claim 1, wherein the individualized assessment protocol dynamically adapts based on previous user responses, modifying subsequent evaluation sessions algorithmically such that once a specific cognitive ability is satisfactorily assessed, the session concludes and transitions to the next task.

13. The method of claim 1, further comprising:
determining, by the server, whether the individualized assessment protocol is completed;
upon determining that the individualized assessment protocol is not completed, continuing the assessment with additional tasks;
upon determining that the individualized assessment protocol is completed, transmitting a request for assessment to the server;
upon receiving the request for assessment, retrieving, by the server, previous individualized assessment scores and latest individualized performance scores;
computing, by the neural network classifier and regression models, individualized assessment scores to generate a profile of cognitive mental abilities; and
transmitting the profile of cognitive mental abilities to the user device.

14. The method of claim 1, further comprising:
storing individualized assessment data in a population database;
processing the data using a neural network classifier and regression models within a communications environment using wired or wireless encrypted protocols; and
generating and transmitting a profile of cognitive mental abilities to the user device.

15. A system comprising a processor and memory, wherein the processor and memory in combination are operable to implement a method comprising:
generating, by a computing system, an individualized assessment protocol comprising a plurality of tasks, wherein each task configured to evaluate at least one cognitive mental specification;
transmitting individualized assessment protocol to a user device;
user selecting at least one task from the plurality of tasks using a semi-random process;
displaying a task-specific interface to present the selected task to the user;
receiving user responses to the task along with associated parameters, including timestamps;
scoring the received user responses based on predefined scoring criteria to generate first individualized performance scores;
processing, by a server, the individualized performance scores using a population database, a neural network classifier, and regression models, which adjust their parameters based on the received scores;
computing, by the neural network classifier and regression models, revised individualized assessment scores to generate a profile of cognitive mental abilities; and
transmitting the profile of cognitive mental abilities to the user device.

16. The system of claim 15, wherein the plurality of tasks includes tasks targeting a different cognitive mental ability, the tasks comprising: game-based challenges involving ordered and/or randomized sequences of subtasks assessing cognitive mental abilities including:
auditory memory, the assessment of auditory memory includes displaying variant numeric keypads with differing key geometries, positions, and orientations, and receiving user responses for evaluation;
auditory and visual discrimination, wherein the assessment of auditory and visual discrimination includes presenting an interface with talking character metaphors, symbolized by non-linguistic characters, existing objects associated with specific sounds, loudspeakers, abstract objects, geometric shapes, or creatures, and receiving user responses for evaluation;
sequencing ability, wherein the assessment of sequencing ability includes presenting an interface displaying dissected objects or images representing a story with a beginning, middle, and end, and receiving user responses for evaluation;
visual memory, wherein the assessment of visual memory includes presenting an interface displaying a two-dimensional array of rectangles, squares, cubes, or polygons, which may have varying arrangements, missing elements, overlapping components, abstract designs, colors, or symbols, wherein users match identical, similar, or related images to complete the task; and
lateralization ability, wherein the assessment of lateralization ability includes presenting an interface displaying an abstract person facing a shelf with objects on the left and right, requiring the user to select an object and the corresponding arm to complete the task.

17. The system of claim 15, wherein the computing comprising:
transmitting, by the server, second individualized performance scores to neural network classifier and regression models; and
comparing the first and second individualized assessment scores by the neural network classifier and regression models to generate the revised individualized assessment scores;
wherein when the second individualized assessment scores are not available, then the first individualized assessment scores is determined to be the revised individualized assessment scores.

18. The system of claim 15, the method further comprising:
storing individualized assessment data in the population database;
processing the data using a neural network classifier and regression models within a communications environment using wired or wireless encrypted protocols; and
generating and transmitting a profile of cognitive mental abilities to the user device.

19. The system of claim 15, the method further comprising:
determining, by the server, whether the individualized assessment protocol is completed;
upon determining that the individualized assessment protocol is not completed, continuing the assessment with additional tasks;
upon determining that the individualized assessment protocol is completed, transmitting a request for assessment to the server;
upon receiving the request for assessment, retrieving, by the server, previous individualized assessment scores and latest individualized performance scores;
computing, by the neural network classifier and regression models, individualized assessment scores to generate a profile of cognitive mental abilities; and
transmitting the profile of cognitive mental abilities to the user device.
